(19) United States
(12) Patent Application Publication
(10) Pub. No.: US 2023/0248301 A1
(43) Pub. Date: Aug. 10, 2023

(54) WEARABLE SENSOR-BASED DEVICE FOR PREDICTING, MONITORING, AND CONTROLLING EPILEPSY AND METHODS THEREOF

(71) Applicants: Muhammad Usman Tariq, Abu Dhabi (AE); Abdullah A. Abonamah, Abu Dhabi (AE)

(72) Inventors: Muhammad Usman Tariq, Abu Dhabi (AE); Abdullah A. Abonamah, Abu Dhabi (AE)

(21) Appl. No.: 18/107,839

(22) Filed: Feb. 9, 2023

Related U.S. Application Data
(60) Provisional application No. 63/308,404, filed on Feb. 9, 2022.

(51) Int. Cl. A61B 5/00

(52) U.S. Cl. CPC A61B 5/4094; A61B 5/746; A61B 5/747; A61B 5/6801; A61B 5/7285; A61B 2562/166

(57) ABSTRACT

The present disclosure relates to a device for predicting, detecting, monitoring, and controlling epilepsy and methods thereof. In particular, the present disclosure relates to early detection and control of epileptic seizures using sensor-based device. In some embodiments, the sensor-based device is wearable.

### CROSS REFERENCE TO RELATED APPLICATIONS

This patent application claims the benefit of and priority to provisional U.S. Application No. 63/308,404, filed on 09 Feb. 2022 and entitled “A Wearable Sensor-Based Device for Predicting, Monitoring, and Controlling Epilepsy and Methods Thereof,” which is incorporated herein by reference in its entirety.

### TECHNICAL FIELD

The present disclosure relates to epilepsy. The present disclosure more particularly relates to devices and methods for predicting, monitoring, and controlling epilepsy.

### BACKGROUND

There are some techniques comprising the state-of-the-art, which provide information about the methods of premediated detection only of epileptic seizures. However, none of them reveals any method for monitoring or as a practical method for detection and monitoring of epileptic seizures, which allow them to identify the epileptic seizure at the ease of use or restriction to the patient.

Different problems and issues with conventional devices and methods for detecting, monitoring and controlling epilepsy are the inadequacy of proposed devices for continuous use in daily life, either by the number of EEG electrodes required (at least 16 electrodes) or by the need of intracranial surgical implants; the need for machine learning period from previous EEG data of the specific user or from a given population to determine algorithm parameters before their practical application; finite-time statistical fluctuations and noise may fundamentally confound the predictive power of the Lyapunov exponent of EEG time series; the cited methods are applicable only to certain types of epilepsy that have a focal point of occurrence in the brain; and all existing techniques require a large amount of input data, which overloads data processing and preventing real-time computing. None of the devices are using the prediction, monitoring, and controlling together in any device or computing using the microprocessor.

Therefore, there is a need improved devices and methods which alleviate at least some of the problems outlined herein. The present invention seeks to provide an improved devices and methods for detecting, monitoring, and controlling epilepsy.

### SUMMARY

The Summary of this invention is not intended to identify key features or essential features of the claimed subject matter, nor is it intended to be used as an aid in determining the scope of the claimed subject matter. The claimed invention relates to a system and method supporting a computerized information system. More specifically, the claimed disclosure relates to relates to devices and methods for early detection, prediction, and control of epileptic seizures.

The claimed disclosure provides a solution in order to overcome a problem specifically arising in the realm of diagnosing brain disorders and more specifically neurological brain disorders using epilepsy as an example. The claims provide a high specific yet sensitive, device and method for early detection, prediction, and control of epileptic seizures. The claimed disclosure overcomes the limitations of current detection tools of brain disorders, and more specifically neurological brain disorders using epilepsy as an example and provides other benefits that will become clear to those skilled in the art from the foregoing description.

Accordingly, in one aspect, the embodiments of the present invention provide for a method for monitoring and predicting epilepsy, comprising: determining a first set of one or more body conditions of a subject by one or more sensors; outputting measurements indicative of the first set of one or more body conditions by the one or more sensors; collecting the measurements indicative of the first set of one or more body conditions by a data collection unit; analyzing collected measurements using an algorithm to balance prediction based on the one or more sensors in real-time and determining any change in value of each of the first set of one or more body conditions outside optimal ranges, computing a collective score indicative of the change in value of the first set of one or more body conditions, wherein the collective score predicts in real-time whether the subject is at normal, pre-seizure or active-seizure state; and determining that the subject is at normal stage triggers the one or more sensors to determining a second set of the one or more body conditions of the subject and repeating the same steps continuously in real-time; evaluating that the subject is at the pre-seizure state or the active-seizure state triggers the following two actions: sending an alert to the subject, emergency contacts and/or a healthcare provider, and triggering electric signals to the brain to control the electric signals to the brain to control the epileptic seizures.

In these embodiments, the one or more sensors are structured in a wearable device. In some embodiments, the one or more sensors are positioned at one or more body parts of the subject.

In the embodiments of the present disclosure, the one or more sensors are positioned and configured to sense at least one or more body conditions comprising blood oxygen, acceleration, temperature, angular velocity, electrocardiography (ECG), glucose level, skin conductance, stress levels and pulse beats.

In the embodiments of the present disclosure, the algorithm is updated continuously to match latest technological improvements for early detection of epileptic seizure event.

In some embodiments, the information collected from the one or more sensors is stored in a built-in memory system in the form of continuous data.

In different embodiments, the data from the one or more sensors is further processed through a microprocessor embedded on a computing board for real-time processing.

In the embodiments of the present disclosure, performing real-time Bluetooth and wireless communication based on data retrieved from the one or more sensors and to generate an alert for the user and health care provider.

In another aspect of the present disclosure, a wearable device for the prediction and control of epilepsy, comprising: one or more sensors to determine one or more body conditions of a subject and outputting data received; collection unit to collect the data from the one or more sensors, wherein the data stored in a built-in memory system in the form of continuous data; a microcircuit computing board that provides real-time Bluetooth and wireless communication with the one or more sensors; a micro-SD port for retrieving data; a printed circuit board to connect electronic components to one another in a controlled manner; a server designed to identify and forecast in real-time a pre-epileptic state based on the data provided by the one or more sensors, wherein the server comprises: a data storage configured to store data provided by the one or more sensors related to the subject; an algorithm storage designed to store an algorithm to balance prediction based on the one or more sensors in real-time and determining any change in value of each of the first set of one or more body conditions outside optimal ranges, wherein the algorithm interacts with a machine learning model for the prediction of the pre-epileptic state using data sets related to the subjects; and a computing processor configured to, when the pre-epileptic state is predicted in real-time based on the algorithm is determined, send alert the controlling unit; a controlling unit designed, once receive an alert for the pre-epileptic state, to trigger in real-time: an alert to the subject, emergency contacts and/or a healthcare provider; and electric signals to the brain to control the electric signals to the brain to control the epileptic seizures.

In some embodiments, the one or more sensors comprises a blood oxygen sensor, an accelerator sensor, a temperature sensor, an electrocardiography (ECG) sensor, a glucometer sensor, a gyroscope sensor, a humidity sensor, a galvanic sensor and a pulse sensor. In these embodiments, the one or more sensors are designed to include a master sensor and one or more subsidiary sensors, wherein the master sensor receives data from the subsidiary sensors and transmits this data to the collection unit.

In one embodiment, algorithm is updated continuously to match latest technological improvements for early detection of epileptic seizure event. In some embodiments, an alert system is attached to send messages to emergency contacts in case of possible detection of seizures, wherein the message is audio or text message.

In the embodiments of this disclosure, a rechargeable battery is used to support the optimal functioning of the device continuously for 24/7 monitoring of the sensor values.

In a different aspect of the disclosure, a method for personalized predicting, early detecting, and controlling epileptic seizures in a subject using a wearable sensor-based device, comprising: identifying different parameters indicative of epilepsy in the subject by one or more sensors; outputting sensed data to a collection unit in the form of continuous data; evaluating collected the sensed data using an algorithm to balance prediction in real-time and determining any change in value of each of the different parameters outside optimal ranges, wherein the algorithm interacts with a machine learning model for the prediction of the pre-epileptic state using data sets specifically related to the subject; computing a collective personalized score indicative of the change in value of the different parameters, wherein the collective personalized score predicts in real-time whether the subject is at normal, pre-seizure or active-seizure state; and determining that the subject is at normal stage triggers the one or more sensors to continuously repeating the same steps in real-time; evaluating that the subject is at the pre-seizure state or the active-seizure state triggers the following two actions: sending an alert to the subject, emergency contacts and/or a healthcare provider, and triggering electric signals to the brain to control the electric signals to the brain to control the epileptic seizures.

In some embodiments, the one or more sensors are positioned at one or more body parts of the subject. In these embodiments, the one or more sensors positioned and configured to sense the different parameters comprising blood oxygen, acceleration, temperature, angular velocity, electrocardiography (ECG), glucose level, skin conductance, stress levels and pulse beats.

In the embodiments of this disclosure, the algorithm is updated continuously to match latest technological improvements by artificial intelligence and machine learning for early detection of epileptic seizure event.

In some embodiments, the data from the one or more sensors is further processed through a microprocessor embedded on a computing board for real-time processing.

In the embodiments of the present disclosure, performing real-time Bluetooth and wireless communication based on data retrieved from the one or more sensors and to generate an alert for the user and health care provider.

Hereinafter the different embodiments and aspects of the present invention is described in detail, however the scope of the present invention should not be restricted to these descriptions, even with the addition to the following examples as appropriate without departing from the spirit of the present invention it may change implementation.

### BRIEF DESCRIPTION OF THE DRAWINGS

Illustrative embodiments of the present invention are described in detail below with reference to the attached drawing figures, and wherein:

FIG. 1 illustrates a diagram of an example device suitable for operation of an embodiment.

### DETAILED DESCRIPTION

It is an object of the present invention to provide superior tools for predicting, monitoring, and controlling a brain disorder, with high specificity and sensitivity. The embodiments of the present disclosure provide a device and method to monitor, predict and control a brain disorder in a subject. More particularly, the embodiments of the present disclosure provide for a device and method for early detection and prediction of a brain disorder using a wearable sensor-device. In some embodiments, the brain disorder is a neurological brain disorder. In other embodiments, the brain disorder is epilepsy.

Epilepsy is a widespread neurological brain disorder that can occur at any age, causing a range of symptoms and is ranked as the second most severe condition following a stroke. It affects millions of individuals globally.

The embodiments of the present disclosure provide for devices and methods for the early detection, prediction, and control of epileptic seizures. The early detection is based on readings from various sensors on a wearable device, such as sensors for blood oxygen, temperature, accelerometer, gyroscope, electrocardiography (ECG), pulse, and glucose levels. The sensors are designed and structured to provide real-time Bluetooth and wireless communication. The device has an internal processor to perform real-time analysis of the sensor data predicting any changes in optimal values, such as temperature or blood oxygen levels, that may indicate an upcoming seizure in a personalized model to the subject. In the event of an imminent seizure, the system generates an alert to the subject, emergency contacts and/or healthcare provider; and may also send electric signals to control the seizure. The major advantage of this disclosure is its ability to provide real-time personalized computing for early detection, prediction, and control of epileptic seizures.

Collectively, the embodiments of the present disclosure provide for a solution that overcomes the limitations and shortfalls of the current systems and methods for the prediction, detection and monitoring of epileptic seizures. Moreover, the present disclosure overcomes the issue of identifying the epileptic seizure at the ease of use or restriction to the patient.

In a first aspect, the embodiments of the present disclosure provide for a method for monitoring and predicting epilepsy, comprising: determining different body conditions of a subject by using a set of sensors to calculate a collective score indicative of the change in value of the different body conditions to predict whether the subject is at normal, pre-seizure or active-seizure state.

In a second aspect, the embodiments of the present disclosure provide for a wearable device for predicting and controlling epilepsy, which includes various sensors to monitor the subject’s body conditions and output data, a microcircuit computing board for real-time communication with the sensors, a micro-SD port for data retrieval, and a server for real-time prediction of pre-epileptic states based on data from the sensors. The server has a data storage, algorithm storage, and computing processor, which interacts with a machine learning model to predict a pre-epileptic state and send an alert to a controlling unit. The controlling unit, upon receiving the alert, triggers real-time warning, as well as electric signals to the brain to control the predicted epileptic seizures.

In a third aspect, the embodiments of the present disclosure provide for a method for predicting, detecting, and controlling epileptic seizures in a subject uses a wearable sensor-based device. The method identifies different parameters indicative of epilepsy in the subject, outputs the data to a collection unit, and evaluates the data using an algorithm in real-time. The algorithm computes a personalized collective score to determine whether the subject at a normal, pre-seizure or active-seizure state. If the subject is in pre-seizure or active-seizure state, a warning alert is sent, and electric signals are triggered to control the epileptic seizures.

Having briefly illustrated embodiments of the present disclosure, it is the aim of one of the aspects to provide for a method for monitoring and predicting epilepsy. The method involves the determination of one or more body conditions of the subject by sensors, outputting of the measurements, collection of the measurements by a data collection unit, and analyzing the collected data using an algorithm. The algorithm balances prediction in real-time and determines if any of the body conditions are outside of optimal ranges. A collective score is computed based on the change in value of the body conditions, which predicts whether the subject is at normal, pre-seizure or active-seizure state. If the subject is at normal state, the process repeats continuously, while if the subject is at pre-seizure or active-seizure state, alerts are sent to the subject, emergency contacts and/or a healthcare provider, and electric signals are triggered to control the epilepsy.

The computing processor will take data from each sensor and check against the threshold change in the optimal values for the sensors. Such as, the temperature sensor has optimal value of 36.5-37.5 Celsius. The computing processor will monitor these values in real-time to predict any change above the ranges. The blood oxygen sensor has an optimal value of 80-100 mm Hg. Once the value drops below the threshold, the computing processor will generate the alert. The accelerometer values are based on the sample’s values with a range of +/- 6 g which depends on each person’s ability and the activities involved. The gyroscope works with an ideal value equal to 1. It is further monitored by the processor for different turning point values. The pulse sensor works on the optimal value of threshold and sensitivity that is adjusted between different numbers from 0-1024. The computation processor will monitor and adjust according to each person. The glucometer sensor is based on the range of 5-10% difference from the optimal values. The ECG sensor is working on optimal value ranges and deviations which are used to calculate the heart rate variability. All values from the sensors are sent to computation unit which processes them in real-time to predict any change in value for epilepsy detection and prediction. Once the predicted value is above the ranges, considered as pre-epileptic alert, the system will send signals for alerting the user, subject, emergency contacts or healthcare provider. In case of an epileptic seizure has already started, the device sends electric signals to the brain to control the incoming predicted epileptic seizure.

In some embodiments of the present disclosure, the sensors of the invention comprise sensors for blood oxygen, accelerometer, temperature, gyroscope, ECG, glucometer, and pulse sensor. In some embodiments, the system comprises a galvanic sensor response is used to measure stress levels that are not linked with real-time detection of epileptic seizures. The galvanic sensor utilizes the skin conductance which is known to be directly involved in the emotional behavioral regulations in humans. A skilled person of the art knows that additional sensors may be added to the invention. In some embodiments the sensors data will be stored in the built-in memory system in the form of continuous data. In some embodiments, the sensor data will be further sent for processing through the microprocessor embedded on the computing board for real-time processing of the inputs from the sensors.

In these embodiments, the one or more sensors are structured in a wearable device. In some embodiments, the one or more sensors are positioned at one or more body parts of the subject. It is known to a skilled person in the art that the device can be worn in one or more parts of the body. In some embodiments, the wearable device with the one or more sensors is wearable on hand, arm, head, ear or any other part of the body.

It is also known that there may be a master sensor with different subsidiary sensors, wherein the master sensor collects all the data from the subsidiary sensors and output the data to the collection unit for processing.

In some embodiments, the method comprises printed circuit board (PCB) for internal computing (is a medium used in electrical and electronic engineering to connect electronic components to one another in a controlled manner). PCB is a customized board designed for the particular invention. In some embodiments, the system combines all sensors in link with the PCB board. In some embodiments, the system comprises collaborative sensory calculation which combines data from the above-mentioned sensors.

In some embodiments, a customized algorithm will predict the value in comparison with each sensor. The algorithm will balance the prediction based on the sensors and further the generation of alerts for the user. This is based on the data collected from above mentioned sensors. The algorithm is updated with the latest artificial intelligence and machine learning as responsive to the technological change. The sensors model also change with the technological change that will be continuously updated to provide the best performance. In some embodiments, the information collected from the one or more sensors is stored in a built-in memory system in the form of continuous data.

In different embodiments, the data from the one or more sensors is further processed through a microprocessor embedded on a computing board for real-time processing.

In the embodiments of the present disclosure, performing real-time Bluetooth and wireless communication based on data retrieved from the one or more sensors and to generate an alert for the user and health care provider. The Bluetooth and wireless communication will communicate the predicted data to the user computer or cloud server. In some embodiments, the predicted data will be sent back to the built-in storage on the customized board for the invention. The method of the present invention is predicting, detecting, and monitoring the information stored through the sensors in real-time with an accuracy of 70%, 80%, 85 %, 90%, or 95%.

In the present disclosure, the information processing is done in real-time parallel to all connected sensors that allow controlling of the pre-seizure, active-seizure, and post-seizure conditions. The controlling is based on the collected data from the sensors, threshold monitoring, prediction of possible seizure through computing processor with electronic signals sent to the brain to control the possible seizures along with generating pre, active, and post-seizure alerts. The advanced approach used by the present disclosure is based on connection with the body that is imperative to get the information.

In some embodiments, the monitoring of the epileptic state is extended to 7 days and 24/7, which is not available in any of the prior art. In some embodiments, an alert system is attached to send messages to the emergency contacts in case of possible prediction or detection of seizures. The continuous working of sensors is vital as the seizures occur due to a combination of various issues that leverage real-time monitoring of all vitals from sensors.

In some embodiments, the information collected from the sensors will be stored in the built-in memory system in the form of continuous data. In some embodiments, the sensors data will be further sent for processing through the microprocessor embedded on the computing board for real-time processing of the inputs from the sensors. In these embodiments, the important aspect of data processing is based on the customized algorithm that will predict the value in comparison with each sensor. The algorithm will balance the prediction based on the sensors and further the generation of alerts for the user. The prediction part is vital as it includes identifying and storing values from the sensor.

In some embodiments, the advanced approach of the methods of the present invention is based on connection with the body that is imperative to get the information. The data gathering from the various sensors is difficult to compute in parallel but has been done using customized algorithms through the sensor-based device of the present invention. The parallel processing further helps to control the seizure at early stages.

In a different aspect of the present disclosure, a wearable device for the prediction and control of epilepsy, comprising: one or more sensors to determine one or more body conditions of a subject and outputting data received; collection unit to collect the data from the one or more sensors, wherein the data stored in a built-in memory system in the form of continuous data; a microcircuit computing board that provides real-time Bluetooth and wireless communication with the one or more sensors; a micro-SD port for retrieving data; a printed circuit board to connect electronic components to one another in a controlled manner; a server designed to identify and forecast in real-time a pre-epileptic state based on the data provided by the one or more sensors, wherein the server comprises: a data storage configured to store data provided by the one or more sensors related to the subject; an algorithm storage designed to store an algorithm to balance prediction based on the one or more sensors in real-time and determining any change in value of each of the first set of one or more body conditions outside optimal ranges, wherein the algorithm interacts with a machine learning model for the prediction of the pre-epileptic state using data sets related to the subjects; and a computing processor configured to, when the pre-epileptic state is predicted in real-time based on the algorithm is determined, send alert the controlling unit; a controlling unit designed, once receive an alert for the pre-epileptic state, to trigger in real-time: an alert to the subject, emergency contacts and/or a healthcare provider; and electric signals to the brain to control the electric signals to the brain to control the epileptic seizures.

In some embodiments, the one or more sensors comprises a blood oxygen sensor, an accelerator sensor, a temperature sensor, an electrocardiography (ECG) sensor, a glucometer sensor, a gyroscope sensor, a humidity sensor, a galvanic sensor and a pulse sensor. In these embodiments, the one or more sensors are designed to include a master sensor and one or more subsidiary sensors, wherein the master sensor receives data from the subsidiary sensors and transmits this data to the collection unit.

It is known to a skilled person in the art that the device can be worn in one or more parts of the body. In some embodiments, the wearable device with the one or more sensors is wearable on hand, arm, head, ear or any other part of the body.

In one embodiment, algorithm is updated continuously to match latest technological needs for early detection of epileptic seizure event. In some embodiments, an alert system is attached to send messages to emergency contacts in case of possible detection of seizures, wherein the message is audio or text message.

In the embodiments of this disclosure, a rechargeable battery is used to support the optimal functioning of the device continuously for 24/7 monitoring of the sensor values. In some embodiments, the device is further connected with a built-in rechargeable battery for up to 7 days. In other embodiments, the device further can be linked with an external display using available USB ports on the device.

In some embodiments, the information processing is done in real-time parallel to all connected sensors that allow controlling of the pre-seizure, active-seizure, and post-seizure conditions. The controlling part was observed missing in the field. The present invention provides accuracy and precision for controlling the pre- and post-seizure conditions effectively.

In some embodiments, the device comprises PCB for internal computing. PCB is a customized board designed for the invention. In some embodiments, the device combines all sensors in link with the PCB board. In some embodiments, the system comprises collaborative sensory calculation which combines data from the above-mentioned sensors.

There can be an additional attachment of sensors using the micro-USB port. It has an internal antenna to pick up the wireless LAN. The device can be powered through a micro-battery and using external power via a micro-USB port.

The device of the present invention is predicting, detecting, and monitoring the information stored through the sensors in real-time with an accuracy of 70%, 80%, 85 %, 90%, or 95%.

In an aspect of the disclosure, a method for personalized predicting, early detecting, and controlling epileptic seizures in a subject using a wearable sensor-based device, comprising: identifying different parameters indicative of epilepsy in the subject by one or more sensors; outputting sensed data to a collection unit in the form of continuous data; evaluating collected the sensed data using an algorithm to balance prediction in real-time and determining any change in value of each of the different parameters outside optimal ranges, wherein the algorithm interacts with a machine learning model for the prediction of the pre-epileptic state using data sets specifically related to the subject; computing a collective personalized score indicative of the change in value of the different parameters, wherein the collective personalized score predicts in real-time whether the subject is at normal, pre-seizure or active-seizure state; and determining that the subject is at normal stage triggers the one or more sensors to continuously repeating the same steps in real-time; evaluating that the subject is at the pre-seizure state or the active-seizure state triggers the following two actions: sending an alert to the subject, emergency contacts and/or a healthcare provider, and triggering electric signals to the brain to control the electric signals to the brain to control the epileptic seizures.

In some embodiments, the one or more sensors are positioned at one or more body parts of the subject. In these embodiments, the one or more sensors positioned and configured to sense the different parameters comprising blood oxygen, acceleration, temperature, angular velocity, electrocardiography (ECG), glucose level, skin conductance, stress levels and pulse beats. It is known to a skilled person in the art that the device can be worn in one or more parts of the body. In some embodiments, the wearable device with the one or more sensors is wearable on hand, arm, head, ear or any other part of the body.

In the embodiments of this disclosure, the algorithm is updated continuously to match latest technological needs by artificial intelligence and machine learning for early detection of epileptic seizure event. In some embodiments, the data from the one or more sensors is further processed through a microprocessor embedded on a computing board for real-time processing.

In the embodiments of the present disclosure, performing real-time Bluetooth and wireless communication based on data retrieved from the one or more sensors and to generate an alert for the user and health care provider.

The method of the present invention is predicting, detecting, and monitoring the information stored through the sensors in real-time with an accuracy of 70%, 80%, 85 %, 90%, or 95%.

In the embodiments of this disclosure, a rechargeable battery is used to support the optimal functioning of the device continuously for 24/7 monitoring of the sensor values. In some embodiments, the device is further connected with a built-in rechargeable battery for up to 7 days. In other embodiments, the device further can be linked with an external display using available USB ports on the device.

The major advantage of the present disclosure that it provides for a device and method thereof for real-time personalized computing for early detection, prediction and controlling the occurrence of epileptic seizures in a subject in need.

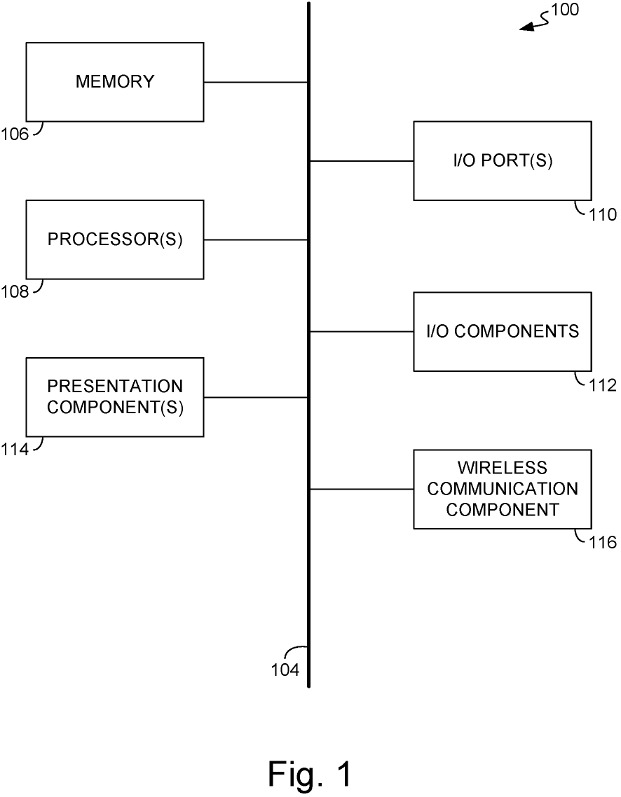

Turning to FIG. 1, it depicts a block diagram of a device 100 suitable to implement embodiments of the present invention. It will be understood by those of ordinary skill in the art that the device 100 is just one non-limiting example of a suitable device and is not intended to limit the scope of use or functionality of the present invention. Similarly, the device 100 should not be interpreted as imputing any dependency and/or any requirements with regard to each component and combination(s) of components illustrated in FIG. 1. It will be appreciated by those having ordinary skill in the art that the connections illustrated in FIG. 1 may comprise other methods, hardware, software, and/or devices for establishing a communications link between the components, devices, systems, and entities. Although the connections are depicted using one or more solid lines, it will be understood by those having ordinary skill in the art that the connections of FIG. 1 may be hardwired or wireless, and may use intermediary components that have been omitted or not included in FIG. 1 for simplicity’s sake. As such, the absence of components from FIG. 1 should be not be interpreted as limiting the present invention to exclude additional components and combination(s) of components. Moreover, though devices and components are represented in FIG. 1 as singular devices and components, it will be appreciated that some embodiments may include a plurality of the devices and components such that FIG. 1 should not be considered as limiting the number of a devices or components.

Continuing, the device 100 may be in the form of a server, in some embodiments. Although illustrated as one component in FIG. 1, the present invention may utilize a plurality of local servers and/or remote servers in the device 100. The server may include components such as a processing unit, internal system memory, and a suitable system bus for coupling to various components, including a database or database cluster. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The server may include or may have access to computer-readable media. Computer-readable media may be any available media that may be accessed by server. Computer-readable media may include one or more of volatile media, nonvolatile media, removable media, or non-removable media. By way of a non-limiting example, computer-readable media may include computer storage media and/or communication media. Non-limiting examples of computer storage media may include one or more of volatile media, nonvolatile media, removable media, or non-removable media, may be implemented in any method and/or any technology for storage of information, such as computer-readable instructions, data structures, program modules, or other data. In this regard, non-limiting examples of computer storage media may include Random Access Memory (RAM), Read-Only Memory (ROM), Electrically Erasable Programmable Read-Only Memory (EEPROM), flash memory or other memory technology, CD-ROM, digital versatile disks (DVDs) or other optical disk storage, magnetic cassettes, magnetic tape, magnetic disk storage, or other magnetic storage device, or any other medium which may be used to store information and which may be accessed by the server. Generally, computer storage media is non-transitory such that it does not comprise a signal per se.

Communication media may embody computer-readable instructions, data structures, program modules, and/or other data in a modulated data signal, such as a carrier wave or other transport mechanism. Communication media may include any information delivery media. As used herein, the term “modulated data signal” refers to a signal that has one or more of its attributes set or changed in such a manner as to encode information in the signal. Non-limiting examples of communication media may include wired media, such as a wired network connection, a direct-wired connection, and/or a wireless media, such as acoustic, radio frequency (RF), infrared, and other wireless media. Combinations of any of the above also may be included within the scope of computer-readable media.

Continuing to FIG. 1, the a block diagram of a device 100 suitable for providing packing instructions is provided, in accordance with an embodiment of the technology. It should be noted that although some components depicted in FIG. 1 are shown in the singular, they may be plural, and the components may be connected in a different, including distributed, configuration. For example, device 100 may include multiple processors and/or multiple sensors. As shown in FIG. 1, device 100 includes a bus 104 that may directly or indirectly connect different components together, including memory 106 and a processor 108. In further embodiments, the device 100 may include one or more of an input/output (I/O) port 110, I/O component 112, presentation component 114, or wireless communication component 116, such as a sensor or other device capable of sensing measurements as previously discussed, and capable of wireless and/or wired transmissions. The device 100 may be coupled to a power supply 118, in some embodiments.

Memory 106 may take the form of the memory components described herein. Thus, further elaboration will not be provided here, but it should be noted that memory 106 may include any type of tangible medium that is capable of storing information, such as a database. A database may include any collection of records, data, and/or other information, such as the algorithm(s) discussed herein and continuous data. In one embodiment, memory 106 may include a set of computer-executable instructions that, when executed, facilitate various functions or steps disclosed herein. These instructions will variously be referred to as “instructions” or an “application” for short. Processor 108 may actually be multiple processors that may receive instructions and process them accordingly. Presentation component 114 may include a display, a speaker, a screen, a portable digital device, and/or other components that may present information through visual (e.g., a display, a screen, a lamp, a light-emitting diode (LED), a graphical user interface (GUI), and/or even a lighted keyboard), auditory (e.g., a speaker), haptic feedback, and/or other tactile cues. Additionally or alternatively, presentation component 114 may include the ability to generate and communicate alerts as previously discussed herein. Wireless communication component 116 may facilitate communication with a network as previously described herein. Additionally or alternatively, the wireless communication component 116 may facilitate other types of wireless communications, such as Wi-Fi, WiMAX, LTE, Bluetooth, and others. In various embodiments, the wireless communication component 116 may be configured to concurrently support multiple technologies.

I/O port 110 may take a variety of forms. Exemplary I/O ports may include a USB jack, a stereo jack, an infrared port, a firewire port, and/or other proprietary communications ports. I/O component 112 may comprise one or more sensors, keyboards, microphones, speakers, touchscreens, and/or any other item useable to directly or indirectly input data into the device 100. Power supply 118 may include batteries, fuel cells, and/or any other component that may act as a power source to supply power to device 100 or to other components.

Although internal components of the device 100 are not illustrated for simplicity, those of ordinary skill in the art will appreciate that internal components and their interconnection are present in the device 100 of FIG. 1. Accordingly, additional details concerning the internal construction of the device 100 are not further disclosed herein.

Regarding FIG. 1, it will be understood by those of ordinary skill in the art that the environment(s), system(s), and/or methods(s) depicted are not intended to limit the scope of use or functionality of the present embodiments. Similarly, the environment(s), system(s), and/or methods(s) should not be interpreted as imputing any dependency and/or any requirements with regard to each component, each step, and combination(s) of components or step(s) illustrated therein. It will be appreciated by those having ordinary skill in the art that the connections illustrated the figures are contemplated to potentially include methods, hardware, software, and/or other devices for establishing a communications link between the components, devices, systems, and/or entities, as may be utilized in implementation of the present embodiments. As such, the absence of component(s) and/or steps(s) from the figures should be not be interpreted as limiting the present embodiments to exclude additional component(s) and/or combination(s) of components. Moreover, though devices and components in the figures may be represented as singular devices and/or components, it will be appreciated that some embodiments may include a plurality of devices and/or components such that the figures should not be considered as limiting the number of a devices and/or components.

It is noted that embodiments of the present invention described herein with reference to block diagrams and flowchart illustrations. However, it should be understood that each block of the block diagrams and/or flowchart illustrations may be implemented in the form of a computer program product, an entirely hardware embodiment, a combination of hardware and computer program products, and/or apparatus, systems, computing devices/entities, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some embodiments, retrieval, loading, and/or execution may be performed in parallel such that multiple instructions are retrieved, loaded, and/or executed together. Thus, such embodiments may produce specifically-configured machines performing the steps or operations specified in the block diagrams and flowchart illustrations. Accordingly, the block diagrams and flowchart illustrations support various combinations of embodiments for performing the specified instructions, operations, or steps.

Additionally, as should be appreciated, various embodiments of the present disclosure described herein may also be implemented as methods, apparatus, systems, computing devices/entities, computing entities, and/or the like. As such, embodiments of the present disclosure may take the form of an apparatus, system, computing device, computing entity, and/or the like executing instructions stored on a computer-readable storage medium to perform certain steps or operations. However, embodiments of the present disclosure may also take the form of an entirely hardware embodiment performing certain steps or operations.

As used herein, all headings are simply for organization and are not intended to limit the disclosure in any manner. The content of any individual section may be equally applicable to all sections.

What is claimed is:

1. A method for monitoring and predicting epilepsy, the method comprising:
determining a first set of one or more body conditions of a subject by one or more sensors;
outputting measurements indicative of the first set of one or more body conditions by the one or more sensors;
collecting the measurements indicative of the first set of one or more body conditions by a data collection unit;
analyzing the measurements collected using an algorithm to balance prediction based on the one or more sensors in real-time to determine a change in value of the first set of one or more body conditions outside optimal ranges;
computing a collective score indicative of the change in value of the first set of one or more body conditions, wherein the collective score predicts in real-time whether the subject is at a normal state, a pre-seizure state, or an active-seizure state;
when the subject is at the normal stage based on the collective score, triggering the one or more sensors to determine a second set of the one or more body conditions of the subject and repeating each prior step in the method continuously in real-time; and
when the subject is at the pre-seizure state or the active-seizure state, triggering:
sending an alert to one or more of the subject, emergency contacts, or a healthcare provider; and
triggering electric signals to a brain of the subject to control the epileptic seizures.

2. The method of claim 1, wherein the one or more sensors are structured in a wearable device.

3. The method of claim 1, wherein the one or more sensors are positioned at one or more body parts of the subject.

4. The method of claim 1, wherein the one or more sensors are positioned and configured to sense at least one of the one or more body conditions including one or more of blood oxygen, acceleration, temperature, angular velocity, electrocardiography (ECG), glucose level, skin conductance, stress levels, or pulse beats.

5. The method of claim 1, wherein the algorithm is updated continuously to match latest technological needs for early detection of epileptic seizure event.

6. The method of claim 1, wherein the measurements collected from the one or more sensors is as data stored in a built-in memory system in the form of continuous data.

7. The method of claim 1, wherein the measurements from the one or more sensors is further processed as data through a microprocessor embedded on a computing board for real-time processing.

8. The method of claim 1, wherein performing real-time Bluetooth and wireless communication based on data from the one or more sensors and to generate an alert for the subject and health care provider.

9. A wearable device for the prediction and control of epilepsy, the device comprising:
one or more sensors to determine one or more body conditions of a subject and outputting data;
a collection unit to collect the data output from the one or more sensors, wherein the data is stored in a built-in memory system in the form of continuous data;
a microcircuit computing board that provides real-time Bluetooth and wireless communication with the one or more sensors;
a micro-SD port for retrieving data;
a printed circuit board to connect electronic components to one another in a controlled manner;
a server designed to identify and forecast in real-time a pre-epileptic state based on the data output by the one or more sensors, wherein the server comprises:
a data storage configured to store the data output by the one or more sensors related to the subject;
an algorithm storage designed to store an algorithm to balance prediction based on the one or more sensors in real-time and to determine a change in value of the first set of one or more body conditions outside optimal ranges, wherein the algorithm interacts with a machine learning model for prediction of the pre-epileptic state using data sets related to the subject; and
a computing processor configured to, when the pre-epileptic state is predicted in real-time based on the algorithm, send an alert to a controlling unit; and
the controlling unit designed to trigger in real-time with receipt of the alert of the pre-epileptic state:
causing an alert to be sent to one or more of the subject, emergency contacts, or a healthcare provider; and
causing electric signals to be sent to a brain of the subject to control the epileptic seizures.

10. The device of claim 9, wherein the one or more sensors comprise one or more of a blood oxygen sensor, an accelerator sensor, a temperature sensor, an electrocardiography (ECG) sensor, a glucometer sensor, a gyroscope sensor, a humidity sensor, a galvanic sensor, or a pulse sensor.

11. The device of claim 9, wherein the one or more sensors are designed to include a master sensor and one or more subsidiary sensors, wherein the master sensor receives data from the subsidiary sensors and transmits the data to the collection unit.

12. The device of claim 9, wherein the algorithm is updated continuously to match latest technological needs for early detection of epileptic seizure event.

13. The device of claim 9, wherein the system further compresses an alert system to send a message to the emergency contacts based on predicted detection of seizures, wherein the message is an audio or a text message.

14. The device of claim 9, wherein a rechargeable battery is used to support the optimal functioning of the device continuously for monitoring of the sensor values 24 hours per day and seven days per week.

15. A method for personalized predicting, early detecting, and controlling epileptic seizures in a subject using a wearable sensor-based device, the method comprising:
identifying different parameters indicative of epilepsy in the subject by one or more sensors;
outputting data from the one or more sensors to a collection unit as continuous data;
evaluating the data using an algorithm to balance prediction in real-time and to determine a change in value of the different parameters that is outside optimal ranges, wherein the algorithm interacts with a machine learning model for the prediction of the pre-epileptic state using data sets specifically related to the subj ect;
computing a collective personalized score indicative of the change in value of the different parameters, wherein the collective personalized score predicts in real-time whether the subject is at a normal state, a pre-seizure state, or an active-seizure state;
determining that the subject is at the normal stage and in response, triggering the one or more sensors to continuously repeat each prior step of the method in real-time; and
determining that the subject is at the pre-seizure state or the active-seizure state and in response, triggering:
sending an alert to one or more of the subject, emergency contacts, or a healthcare provider; and
triggering electric signals to a brain of the subject to control epileptic seizures.

16. The method of claim 15, wherein the one or more sensors are positioned at one or more body parts of the subject.

17. The method of claim 15, wherein the one or more sensors are positioned and configured to sense the different parameters comprised of one or more of blood oxygen, acceleration, temperature, angular velocity, electrocardiography (ECG), glucose level, skin conductance, stress levels, or pulse beats.

18. The method of claim 15, wherein the algorithm is updated continuously to match latest technological needs by artificial intelligence and machine learning for early detection of an epileptic seizure event.

19. The method of claim 15, wherein the data output by the one or more sensors is further processed through a microprocessor embedded on a computing board for real-time processing.

20. The method of claim 15, wherein performing real-time Bluetooth and wireless communication based on the data output by the one or more sensors and to generate the alert for the subject and a health care provider.
